(19) United States
(12) Patent Application Publication
(10) Pub. No.: US 2017/0286786 A1
(43) Pub. Date: Oct. 5, 2017

(54) DEVIATION-INDUCED DYNAMIC MODULATION OF IMPULSE RESPONSE FOR DETECTION AND MODELING

(71) Applicant: DAQRI, LLC, Los Angeles, CA (US)

(72) Inventors: Teresa Ann Nick, Woodland Hills, CA (US); Laura Berman, Venice, CA (US); Arye Barnehama, Venice, CA (US)

(21) Appl. No.: 15/089,345

(22) Filed: Apr. 1, 2016

(51) Int. Cl. G06K 9/00; A61B 5/00; G06F 1/16

(52) U.S. Cl. CPC G06K 9/00885; G06F 1/163; A61B 5/0022; A61B 5/6803; A61B 5/742; G06K 2009/00939

(57) ABSTRACT

A wearable computing device includes a display device for displaying augmented reality (AR) images and a biometric monitor for monitoring biometric sensor data obtained by a biometric sensor. The biometric monitor determines whether the biometric data indicates that a user associated with the biometric data could be experiencing a health-related problem. The biometric monitor is configured with various modules to perform deviation-induced dynamic modulation in making this determination.

### TECHNICAL FIELD

The subject matter disclosed herein generally relates to a deviation-induced dynamic modulation of impulse response for detection and modeling and, in particular, to a biometric monitor that leverages the measurements obtained by a biometric sensor to perform the deviation-induced dynamic modulation.

### BACKGROUND

Augmented reality (AR) is a live direct or indirect view of a physical, real-world environment whose elements are augmented (or supplemented) by computer-generated sensory input such as sound, video, graphics or Global Positioning System (GPS) data. With the help of advanced AR technology (e.g., adding computer vision and object recognition) the information about the surrounding real world of the user becomes interactive. Device-generated (e.g., artificial) information about the environment and its objects can be overlaid on the real world.

Typically, a user uses a computing device to view the augmented reality. The computing device may be a wearable computing device used in an environment where the user's health is an important consideration. The computing device may also include a biometric sensor that obtains information about the user's health, such as the user's heartrate. However, a conventional biometric sensor is unable to ascertain whether the measurements it is obtaining are within a user's expected biometric measurements or whether the measurements are indicative of a potential health hazard. Thus, the conventional biometric sensor is unable to predict whether the user is experiencing a problem, such as a cardiac event, which can lead to the user failing to seek medical attention before the problem becomes more severe.

### BRIEF DESCRIPTION OF THE DRAWINGS

Some embodiments are illustrated by way of example and not limited to the figures of the accompanying drawings.

FIG. 1 is a block diagram illustrating an example of a network environment suitable for a wearable computing device, according to an example embodiment.

FIG. 2 is a block diagram of a biometric monitor, according to an example embodiment.

FIG. 3 illustrates a method, according to an example embodiment, implemented by the biometric monitor of FIG. 2 for notifying a user of a potential health problem.

FIGS. 4A-4E further illustrate a method, according to an example embodiment, implemented by the biometric monitor of FIG. 2 for notifying a user of a potential health problem.

FIG. 5 is a block diagram illustrating components of a machine, according to some example embodiments, able to read instructions from a machine-readable medium (e.g., a machine-readable storage medium) and perform any one or more of the methodologies discussed herein.

### DETAILED DESCRIPTION

The disclosure provides for a biometric monitor that includes a machine-readable memory storing computer-executable instructions, and at least one hardware processor in communication with the machine-readable memory that, when the computer-executable instructions are executed, configures the biometric monitor to receive biometric sensor data, determine whether the biometric sensor data is out of an expected range, and in response to the determination that the biometric sensor data is out of an expected range, adjust a first weighting factor by a predetermined amount. The biometric monitor is also configured to determine whether the first weighting factor is out of an expected range, and in response to the determination that the first weighting factor is out of the expected range, increment a counter associated with the first weighting factor. Furthermore, the biometric monitor is configured compute a biometric deviation sensor value based on the first weighting factor, the received biometric sensor data, and a previously computed biometric deviation sensor value, and communicate the computed biometric deviation sensor value via a communication interface communicatively coupled to the at least one hardware processor.

In another embodiment of the biometric monitor, the first weighting factor is adjusted by incrementing the first weighting factor by the predetermined amount.

In a further embodiment of the biometric monitor, the first weighting factor is adjusted by decrementing the first weighting factor by the predetermined amount.

In yet another embodiment of the biometric monitor, the determination that the first weighting factor is out of the expected range comprises comparing the first weighting factor with a minimum weighting factor threshold, and the counter is associated with the minimum weighting factor threshold.

In yet a further embodiment of the biometric monitor, the biometric monitor is further configured to compare the counter with a minimum counter threshold, and based on the comparison of the counter with the minimum counter threshold, execute one or more training operations to train the biometric monitor based on further received biometric sensor data.

In another embodiment of the biometric monitor, the determination that the first weighting factor is out of the expected range comprises comparing the first weighting factor with a maximum weighting factor threshold, and the counter is associated with the maximum weighting factor threshold.

In a further embodiment of the biometric monitor, the biometric monitor is further configured to determine whether the computed biometric deviation sensor value exceeds a maximum biometric deviation sensor value threshold, determine whether the computer biometric deviation sensor value is less than a minimum biometric deviation sensor value threshold, in response to the determination that the computed biometric deviation sensor value exceeds the maximum biometric deviation sensor value threshold, execute at least one notification operation associated with the maximum biometric deviation sensor value threshold, and in response to the determination that the computed biometric deviation sensor value is less than the minimum biometric deviation sensor value threshold, execute at least one notification operation associated with the minimum biometric deviation sensor value threshold, the at least one notification operation associated with the minimum biometric deviation sensor value threshold being different than the at least one notification operation associated with the maximum biometric deviation sensor value threshold.

A method is also disclosed where, in one embodiment, the method includes receiving, by at least one hardware processor, biometric sensor data, determining, by at least one hardware processor, whether the biometric sensor data is out of an expected range, and in response to the determination that the biometric sensor data is out of an expected range, adjusting, by at least one hardware processor, a first weighting factor by a predetermined amount. The method may also include determining, by at least one hardware processor, whether the first weighting factor is out of an expected range, and in response to the determination that the first weighting factor is out of the expected range, incrementing, by at least one hardware processor, a counter associated with the first weighting factor. The method may further include computing, by at least one hardware processor, a biometric deviation sensor value based on the first weighting factor, the received biometric sensor data, and a previously computed biometric deviation sensor value, and communicating, by at least one hardware processor, the computed biometric deviation sensor value via a communication interface communicatively coupled to the at least one hardware processor.

In another embodiment of the method, the first weighting factor is adjusted by incrementing the first weighting factor by the predetermined amount.

In a further embodiment of the method, the first weighting factor is adjusted by decrementing the first weighting factor by the predetermined amount.

In yet another embodiment of the method, the determination that the first weighting factor is out of the expected range comprises comparing the first weighting factor with a minimum weighting factor threshold, and the counter is associated with the minimum weighting factor threshold.

In yet a further embodiment of the method, the method further includes comparing the counter with a minimum counter threshold, and based on the comparison of the counter with the minimum counter threshold, executing one or more training operations to train a biometric monitor based on further received biometric sensor data.

In another embodiment of the method, the determination that the first weighting factor is out of the expected range comprises comparing the first weighting factor with a maximum weighting factor threshold, and the counter is associated with the maximum weighting factor threshold.

In a further embodiment of the method, the method includes determining whether the computed biometric deviation sensor value exceeds a maximum biometric deviation sensor value threshold and determining whether the computer biometric deviation sensor value is less than a minimum biometric deviation sensor value threshold. The method also includes, in response to the determination that the computed biometric deviation sensor value exceeds the maximum biometric deviation sensor value threshold, executing at least one notification operation associated with the maximum biometric deviation sensor value threshold, and in response to the determination that the computed biometric deviation sensor value is less than the minimum biometric deviation sensor value threshold, executing at least one notification operation associated with the minimum biometric deviation sensor value threshold, the at least one notification operation associated with the minimum biometric deviation sensor value threshold being different than the at least one notification operation associated with the maximum biometric deviation sensor value threshold.

This disclosure also contemplates a machine-readable medium storing computer-executable instructions that, when executed by at least one hardware processor, causes a biometric monitor to perform the method and operations described herein.

Unless explicitly stated otherwise, components and functions are optional and may be combined or subdivided, and operations may vary in sequence or be combined or subdivided. In the following description, for purposes of explanation, numerous specific details are set forth to provide a thorough understanding of example embodiments. It will be evident to one skilled in the art, however, that the present subject matter may be practiced without these specific details.

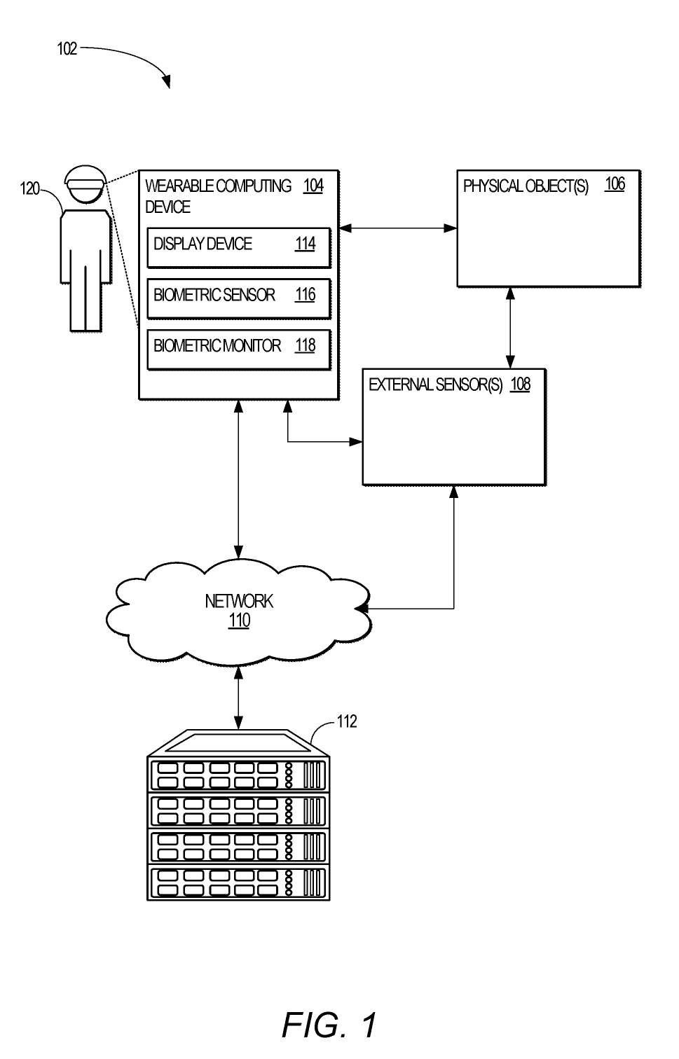

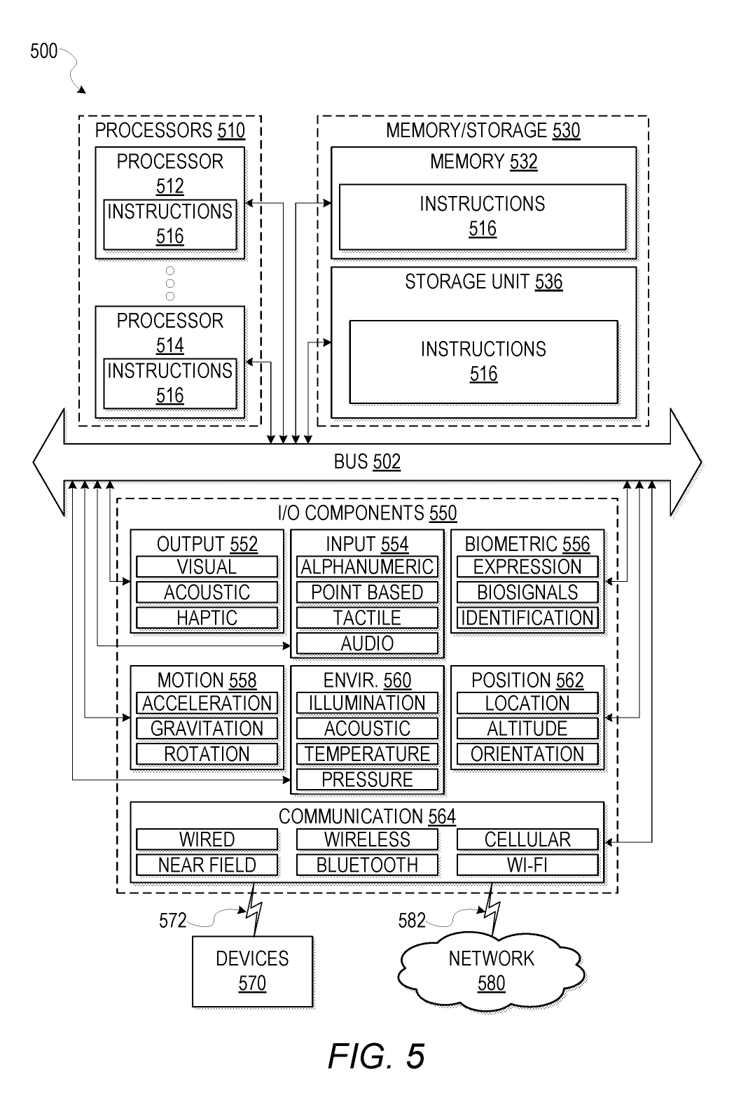

FIG. 1 is a block diagram illustrating an example of a network environment 102 suitable for a wearable computing device 104, according to an example embodiment. The network environment 102 includes the wearable computing device 104 and a server 112 communicatively coupled to each other via a network 110. The wearable computing device 104 further includes a display device 114, a biometric sensor 116, and a biometric monitor 118. The wearable computing device 104 and the server 112 may each be implemented in a computer system, in whole or in part, as described below with respect to FIG. 5.

The server 112 may be part of a network-based system. For example, the network-based system may be or include a cloud-based server system that provides additional information, such as three-dimensional (3D) models or other virtual objects, to the wearable computing device 104.

The wearable computing device 104 may be implemented in various form factors. In one embodiment, the wearable computing device 104 is implemented as a helmet, which the user 120 wears on his or her head, and views objects (e.g., physical object(s) 106) through a display device 114, such as one or more lenses, affixed to the wearable computing device 104. In another embodiment, the wearable computing device 104 is implemented as a lens frame, where the display device 114 is implemented as one or more lenses affixed thereto. In yet another embodiment, the wearable computing device 104 is implemented as a watch (e.g., a housing mounted or affixed to a wrist band), and the display device 114 is implemented as a display (e.g., liquid crystal display (LCD) or light emitting diode (LED) display) affixed to the wearable computing device 104.

A user 120 may wear the wearable computing device 104 and view one or more physical object(s) 106 in a real world physical environment. The user 120 may be a human user (e.g., a human being), a machine user (e.g., a computer configured by a software program to interact with the wearable computing device 104), or any suitable combination thereof (e.g., a human assisted by a machine or a machine supervised by a human). The user 120 is not part of the network environment 102, but is associated with the wearable computing device 104. For example, the wearable computing device 104 may be a computing device with a camera and a transparent display. In another example embodiment, the wearable computing device 104 may be hand-held or may be removably mounted to the head of the user 120. In one example, the display device 114 may include a screen that displays what is captured with a camera (not shown) of the wearable computing device 104. In another example, the display of the display device 114 may be transparent or semi-transparent such as in lenses of wearable computing glasses or the visor or a face shield of a helmet.

The user 120 may be a user of an augmented reality (AR) application executable by the wearable computing device 104 and/or the server 112. The AR application may provide the user 120 with an AR experience triggered by one or more identified objects (e.g., physical object(s) 106) in the physical environment. For example, the physical object(s) 106 may include identifiable objects such as a two-dimensional (2D) physical object (e.g., a picture), a 3D physical object (e.g., a factory machine), a location (e.g., at the bottom floor of a factory), or any references (e.g., perceived corners of walls or furniture) in the real-world physical environment. The AR application may include computer vision recognition to determine various features within the physical environment such as corners, objects, lines, letters, and other such features or combination of features.

In one embodiment, the objects in an image captured by the wearable computing device 104 are tracked and locally recognized using a local context recognition dataset or any other previously stored dataset of the AR application. The local context recognition dataset may include a library of virtual objects associated with real-world physical objects or references. In one embodiment, the wearable computing device 104 identifies feature points in an image of the physical object 106. The wearable computing device 104 may also identify tracking data related to the physical object 106 (e.g., GPS location of the wearable computing device 104, orientation, or distance to the physical object(s) 106). If the captured image is not recognized locally by the wearable computing device 104, the wearable computing device 104 can download additional information (e.g., 3D model or other augmented data) corresponding to the captured image, from a database of the server 112 over the network 110.

In another example embodiment, the physical object(s) 106 in the image is tracked and recognized remotely by the server 112 using a remote context recognition dataset or any other previously stored dataset of an AR application in the server 112. The remote context recognition dataset may include a library of virtual objects or augmented information associated with real-world physical objects or references.

In one embodiment, the wearable computing device 104 also includes a biometric sensor 116 affixed thereto. For example, where the wearable computing device 104 is implemented as a head-mounted device, the biometric sensor 116 may be disposed within an interior surface of the wearable computing device 104 such that the biometric sensor 116 is in contact with the skin of the user's 104 head (e.g., the forehead). As another example, where the wearable computing device 104 is implemented as a wrist-mounted device (e.g., a watch), the biometric sensor 116 may be disposed within, or in contact with, an exterior surface of the wearable computing device 104 such that the biometric sensor 116 is also in contact with the skin of one of the user's 104 limbs (e.g., a wrist of a forearm). In either examples, the biometric sensor 116 is arranged or disposed within the wearable computing device 104 such that it records physiological signals from the user 104.

The biometric sensor 116 is configured to obtain and provide biometric sensor data of the user 120. Examples of the biometric sensor 116 include, but are not limited to, an ocular camera attached to the wearable computing device 104 and directed towards the eyes of the user. In another example, the biometric sensor 116 includes one or more EEG/ECG sensors affixed to an inside surface of the wearable computing device 104 so that the EEG/ECG sensors make contact with the surface of the user when the wearable computing device 104 is worn. The biometric sensor 116 generates biometric data based on the physiological activities of the user 120 including, but not limited to, the blood vessel pattern in the retina of an eye of the user 120, the structure pattern of the iris of an eye of the user 120, the brain wave pattern of the user 120, the heart beat of the user 120, and other such physiological activities.

In one embodiment, the biometric sensor 116 communicates with the display device 114 to display one or more measurements on the display device 114. For example, where the display device 114 is an LED display, the display device 114 may display a resting heart rate obtained from the biometric sensor 116. Further still, where the display device 114 is a lens or other transparent display through which the user 104 views one or more physical object(s) 106, the measurements obtained from the biometric sensor 116 may also be displayed on a lens of the display device 114. Similarly, one or more alerts and notifications generated by the biometric sensor 116 may also be displayed on the display device 114, such as where an irregular heart beat is detected or determined, or where a detected heart beat exceeds (or falls below) a preconfigured heart beat threshold. In these instances, the wearable computing device 104 may be further configured to communicate an alert (e.g., via wireless communication) to a provider of emergency services.

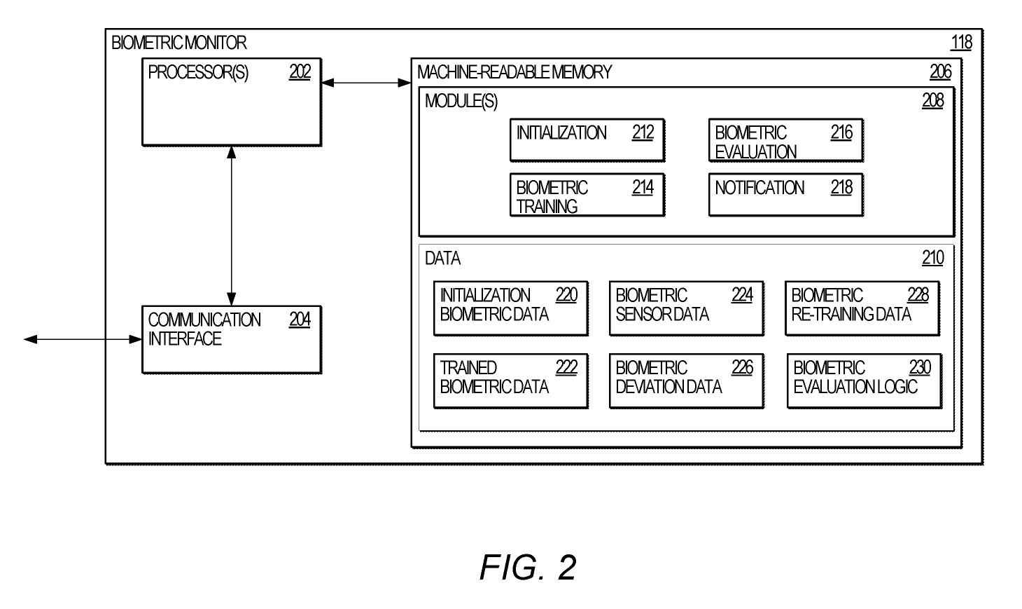

In addition, the wearable computing device 104 includes a biometric monitor 118 configured to monitor the biometric measurements obtained by the biometric sensor 116. In one embodiment, the biometric monitor 118 is configured to maintain a running buffer of one or more prior biometric measurements and to use the running buffer of the one or more prior biometric measurements to determine and/or predict whether the user 120 could be experiencing a problem with an organ associated with the biometric measurement (e.g., the heart, the lungs, etc.). In addition, the biometric monitor 118 is configured with one or more thresholds to reduce and/or eliminate the potential for false positives and/or false negatives. As discussed below with reference to FIG. 2, where the biometric monitor 118 experiences a threshold number of potential false positives and/or false negatives, the biometric monitor 118 may be retrained and/or conditioned using the user's 120 particular biometric measurements. In this regard, while the biometric monitor 118 may be preconfigured with various default thresholds and/or tolerances, the biometric monitor 118 may be further tailored and/or trained to the user's 120 unique physiology.

In one embodiment, the biometric monitor 118 also communicates with the display device 114 to display various notifications and/or measurements obtained from the biometric sensor 116 on the display device 114. For example, where the display device 114 is an LED display, the display device 114 may display a one or more notifications and/or alerts communicated by the biometric monitor 118. Further still, where the display device 114 is a lens or other transparent display through which the user 120 views one or more physical object(s) 106, the output generated by the biometric monitor 118 may also be displayed on a lens of the display device 114. Where the biometric monitor 118 detects a potential health problem of the user 120 in response to the measurements obtained by the biometric sensor 116, the biometric monitor 118 may be further configured to communicate an alert (e.g., via wireless communication) to a provider of emergency services.

The network environment 102 also includes one or more external sensors 108 that interact with the wearable computing device 104 and/or the server 112. The external sensors 108 may be associated with, coupled to, or related to the physical object(s) 106 to measure a location, status, and characteristics of the physical object(s) 106. Examples of measured readings may include but are not limited to weight, pressure, temperature, velocity, direction, position, intrinsic and extrinsic properties, acceleration, and dimensions. For example, external sensors 108 may be disposed throughout a factory floor to measure movement, pressure, orientation, and temperature. The external sensor(s) 108 can also be used to measure a location, status, and characteristics of the wearable computing device 104 and the user 120. The server 112 can compute readings from data generated by the external sensor(s) 108. The server 112 can generate virtual indicators such as vectors or colors based on data from external sensor(s) 108. Virtual indicators are then overlaid on top of a live image or a view of the physical object(s) 106 (e.g., displayed on the display device 114) in a line of sight of the user 120 to show data related to the physical object(s) 106. For example, the virtual indicators may include arrows with shapes and colors that change based on real-time data. Additionally and/or alternatively, the virtual indicators are rendered at the server 112 and streamed to the wearable computing device 104.

The external sensor(s) 108 may include one or more sensors used to track various characteristics of the wearable computing device 104 including, but not limited to, the location, movement, and orientation of the wearable computing device 104 externally without having to rely on sensors internal to the wearable computing device 104. The external senor(s) 108 may include optical sensors (e.g., a depth-enabled 3D camera), wireless sensors (e.g., Bluetooth, Wi-Fi), Global Positioning System (GPS) sensors, and audio sensors to determine the location of the user 120 wearing the wearable computing device 104, distance of the user 120 to the external sensor(s) 108 (e.g., sensors placed in corners of a venue or a room), the orientation of the wearable computing device 104 to track what the user 120 is looking at (e.g., direction at which a designated portion of the wearable computing device 104 is pointed, e.g., the front portion of the wearable computing device 104 is pointed towards a player on a tennis court).

Furthermore, data from the external senor(s) 108 and internal sensors (not shown) in the wearable computing device 104 may be used for analytics data processing at the server 112 (or another server) for analysis on usage and how the user 120 is interacting with the physical object(s) 106 in the physical environment. Live data from other servers may also be used in the analytics data processing. For example, the analytics data may track at what locations (e.g., points or features) on the physical object(s) 106 or virtual object(s) (not shown) the user 120 has looked, how long the user 120 has looked at each location on the physical object(s) 106 or virtual object(s), how the user 120 wore the wearable computing device 104 when looking at the physical object(s) 106 or virtual object(s), which features of the virtual object(s) the user 120 interacted with (e.g., such as whether the user 120 engaged with the virtual object), and any suitable combination thereof. To enhance the interactivity with the physical object(s) 106 and/or virtual objects, the wearable computing device 104 receives a visualization content dataset related to the analytics data. The wearable computing device 104, via the display device 114, then generates a virtual object with additional or visualization features, or a new experience, based on the visualization content dataset.

Any of the machines, databases, or devices shown in FIG. 1 may be implemented in a general-purpose computer modified (e.g., configured or programmed) by software to be a special-purpose computer to perform one or more of the functions described herein for that machine, database, or device. For example, a computer system able to implement any one or more of the methodologies described herein is discussed below with respect to FIG. 5. As used herein, a “database” is a data storage resource and may store data structured as a text file, a table, a spreadsheet, a relational database (e.g., an object-relational database), a triple store, a hierarchical data store, or any suitable combination thereof. Moreover, any two or more of the machines, databases, or devices illustrated in FIG. 1 may be combined into a single machine, and the functions described herein for any single machine, database, or device may be subdivided among multiple machines, databases, or devices.

The network 108 may be any network that facilitates communication between or among machines (e.g., server 110), databases, and devices (e.g., the wearable computing device 104 and the external sensor(s) 108). Accordingly, the network 108 may be a wired network, a wireless network (e.g., a mobile or cellular network), or any suitable combination thereof. The network 108 may include one or more portions that constitute a private network, a public network (e.g., the Internet), or any suitable combination thereof.

FIG. 2 is a block diagram of the components of the biometric monitor 118 according to an example embodiment. In one embodiment, the biometric monitor 118 includes one or more processors 202, a communication interface 204, and a machine-readable memory 206.

The one or more processors 202 may be any type of commercially available processor, such as processors available from the Intel Corporation, Advanced Micro Devices, Qualcomm, Texas Instruments, or other such processors. Further still, the one or more processors 202 may include one or more special-purpose processors, such as a Field-Programmable Gate Array (FPGA) or an Application Specific Integrated Circuit (ASIC). The one or more processors 202 may also include programmable logic or circuitry that is temporarily configured by software to perform certain operations. Thus, once configured by such software, the one or more processors 202 become specific machines (or specific components of a machine) uniquely tailored to perform the configured functions and are no longer general-purpose processors.

The communication interface 204 is configured to facilitate electronic communications between the biometric monitor 118, the biometric sensor 116, the wearable computing device 104, and/or the display device 114. The communication interface 204 may include one or more wired communication interfaces (e.g., Universal Serial Bus (USB), an I²C bus, an RS-232 interface, an RS-485 interface, etc.), one or more wireless transceivers, such as a Bluetooth® transceiver, a Near Field Communication (NFC) transceiver, an 802.11x transceiver, a 3G (e.g., a GSM and/or CDMA) transceiver, a 4G (e.g., LTE and/or Mobile WiMAX) transceiver, or combinations of wired and wireless interfaces and transceivers. In one embodiment, the communication interface 204 communicates data 210, such as the biometric deviation data 226, to the wearable computing device 104 and/or the display device 114. The biometric monitor 118 may also receive instructions and/or biometric sensor data 224 from the wearable computing device 104 or the biometric sensor 116 via the communication interface 204. For example, the biometric sensor 116 may provide the biometric monitor 118 with biometric information about the user 120, such as one or more heartrate measurements, one or more breathing rate measurements, and other such measurements.

The machine-readable memory 206 includes various modules 208 and data 210 for implementing the features of the biometric monitor 118. The machine-readable memory 206 includes one or more devices configured to store instructions and data temporarily or permanently and may include, but not be limited to, random-access memory (RAM), read-only memory (ROM), buffer memory, flash memory, optical media, magnetic media, cache memory, other types of storage (e.g., Erasable Programmable Read-Only Memory (EEPROM)) and/or any suitable combination thereof. The term “machine-readable memory” should be taken to include a single medium or multiple media (e.g., a centralized or distributed database, or associated caches and servers) able to store the modules 208 and the data 210. Accordingly, the machine-readable memory 206 may be implemented as a single storage apparatus or device, or, alternatively and/or additionally, as “cloud-based” storage systems or storage networks that include multiple storage apparatus or devices. As shown in FIG. 2, the machine-readable memory 206 excludes signals per se.

In one embodiment, the modules 208 are written in a computer-programming and/or scripting language. Examples of such languages include, but are not limited to, C, C++, C#, Java, JavaScript, Perl, Python, Ruby, or any other computer programming and/or scripting language now known or later developed.

The modules 208 include one or more modules 212-218 that implement the features of the biometric monitor 118. In one embodiment, the modules include an initialization module 212, a biometric training module 214, a biometric evaluation module 216, and a notification module 218. The data 210 includes one or more different sets of data 220-230 used by, or in support of, the modules 208. In one embodiment, the data 210 includes initialization biometric data 220, trained biometric data 222, biometric sensor data 224, biometric deviation data 226, biometric re-training data 228, and biometric evaluation logic 230.

The initialization module 212 is configured to initialize and establish various values for one or more variables used by the biometric monitor 118 in monitoring the biometric sensor data 224 received from the biometric sensor 116 and/or the wearable computing device 104. In one embodiment, the initialization biometric data 220 includes the values referenced by the initialization module 212 when the biometric monitor 118 is first put into use by the user 120 (e.g., provided with electric power as part of the wearable computing device 104). The initialization module 212 may further assign the values defined by the initialization biometric data 220 in response to one or more resetting actions by the user 120, such as when the user affirmatively resets the wearable computing device 104 and/or the biometric monitor 118 in particular. The initialization module 212 may further load the biometric initialization data 220 in response to a determination that user-specific data (e.g., the trained biometric data 222 and/or the biometric re-training data 228) has become corrupted or is no longer available to the biometric monitor 118.

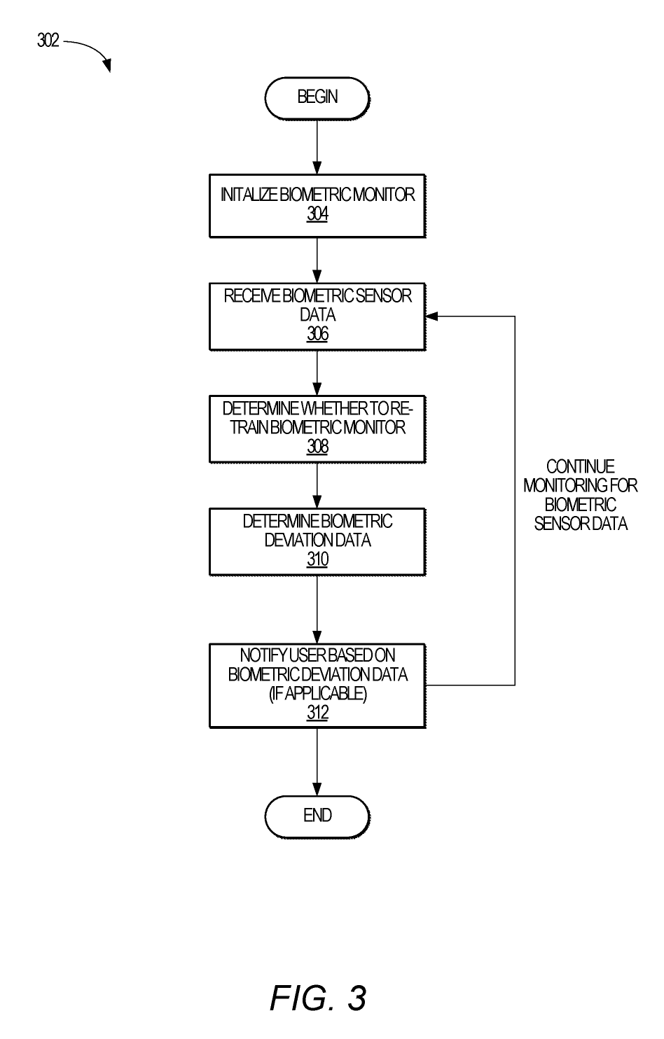

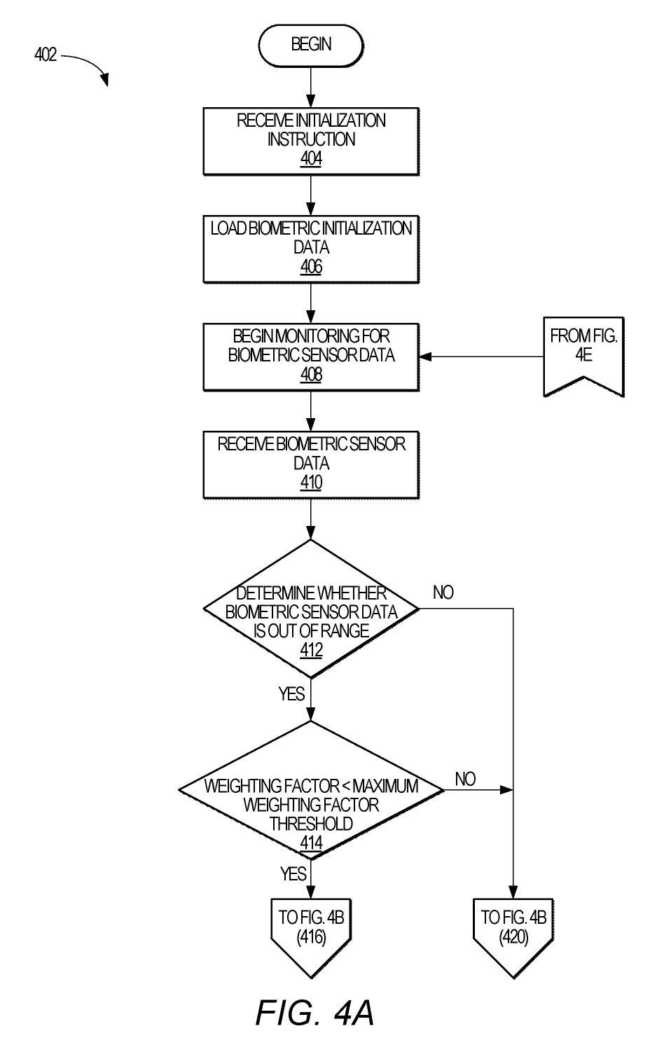

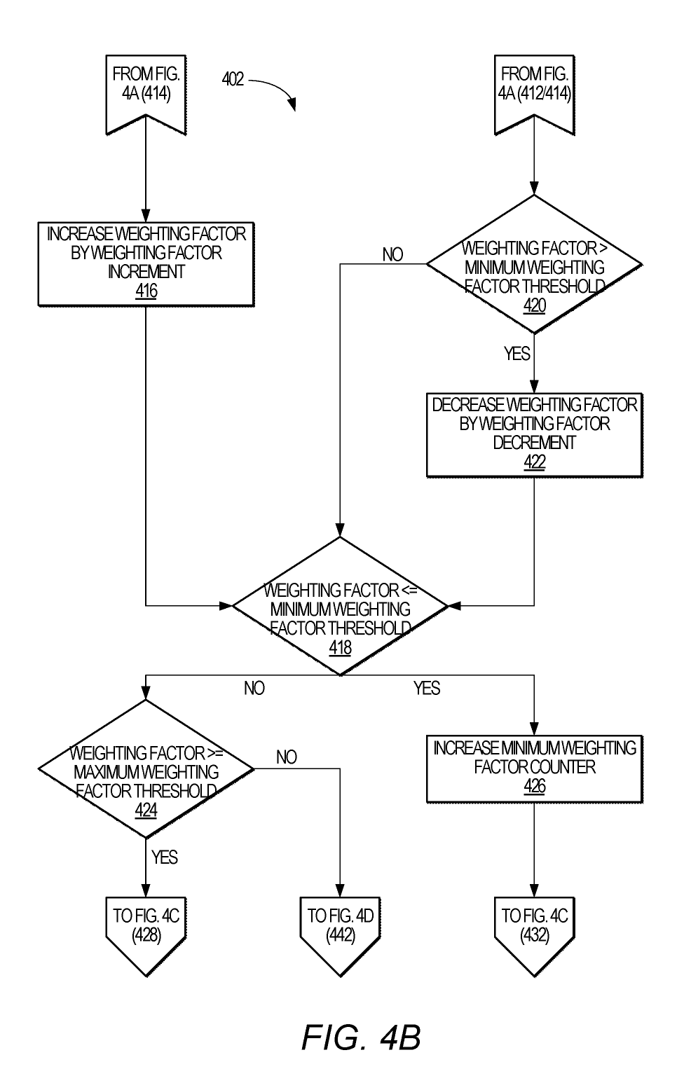

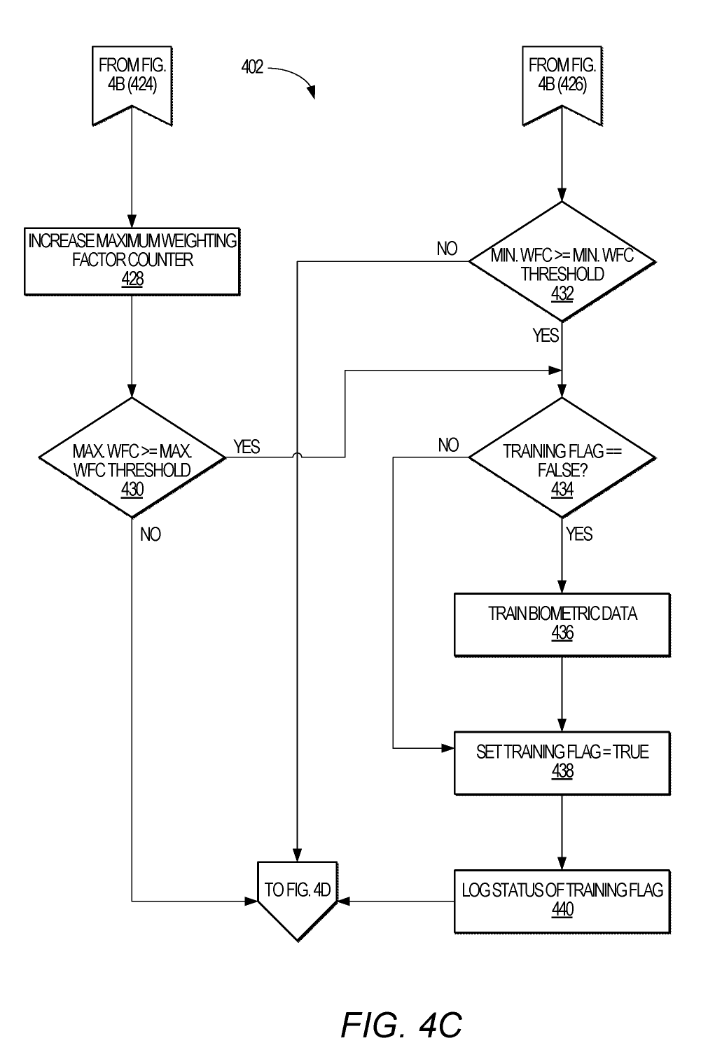

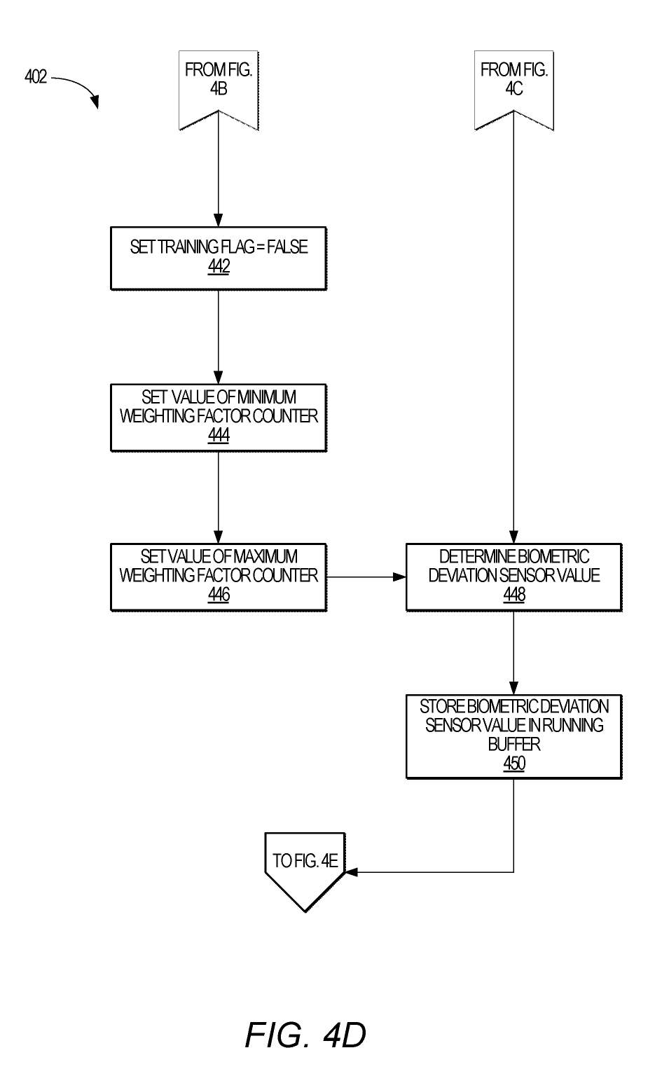

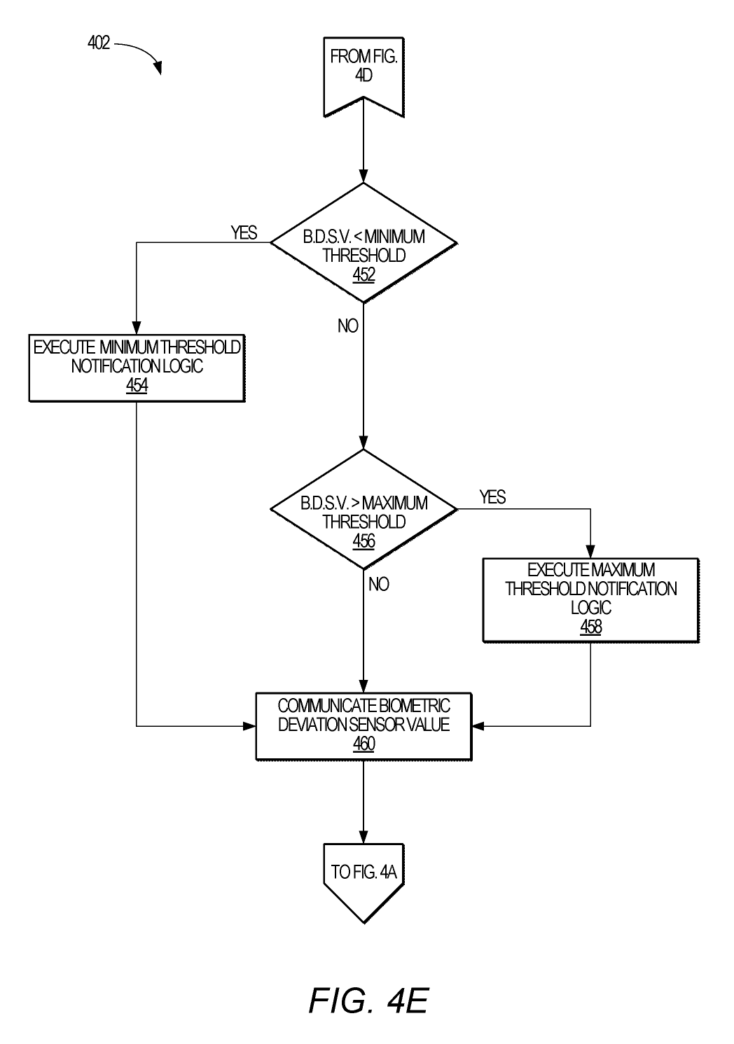

Table 1 below identifies the various variables leveraged by the biometric monitor 118, which are also further discussed with reference to FIGS. 3-4E, and initialization values assigned to the variables (e.g., from the biometric initialization biometric data 220).

TABLE 1

| Variable | Variable | | Initialization |
|---|---|---|---|
| Name | Representation | Description | Value |
| Weighting | α | This is a weighting | Between 0 and 1, |
| Factor | | factor applied to the | such as 0.6. |
| | | received biometric | |
| | | sensor | |
| | | measurement. | |
| Minimum | α_{MIN} | This is a minimum | Between |
| Weighting | | weighting factor | 0 and 1, |
| Factor | | threshold that the | such as 0.35. |
| Threshold | | weighting factor | |
| | | should not go below. | |
| Maximum | α_{MAX} | This is a maximum | Between 0 and 1, |
| Weighting | | weighting factor | such as 0.95. |
| Factor | | threshold that the | |
| Threshold | | weighting factor | |
| | | should not exceed | |
| Minimum | X_{MIN} | This is a minimum | N/A. |
| Biometric | | threshold that the | X_{MIN} can be trained |
| Sensor | | biometric sensor | using the biometric |
| Data | | data should not go | training module 214. |
| Threshold | | below. | |
| Maximum | X_{MAX} | This is a maximum | N/A |
| Biometric | | threshold that the | X_{MAX} |
| Sensor | | biometric sensor | can be trained |
| Data | | data should not | using the biometric |
| Threshold | | exceed. | training module 214. |
| Weighting | α_{INCREMENT} | This is a value by | Between 0 and 1, |
| Factor | | which the weighting | such as 0.05. |
| Increment | | factor is | |
| | | incremented. | |
| Weighting | α_{DECREMENT} | This is a value by | Between 0 and 1. |
| Factor | | which the weighting | This value can be |
| Decrement | | factor is | determined |
| | | decremented. | according to |
| | | | α_{INCREMENT}, |
| | | | such as 0.5 × |
| | | | α_{INCREMENT.} |
| Biometric | X_{T} | This is the biometric | None. |
| Sensor | | measurement | X_{T} is provided |
| Data | | data obtained | by the |
| | | from the | biometric sensor |
| | | biometric sensor at a | 116. |
| | | given time T. | |
| Biometric | X’_{T} | This is a weighted | None. |
| Deviation | | biometric value | This value is |
| Sensor | | determined from | determined |
| Value | | X_{T} and X_{T-1}. | from X_{T} |
| | | | and X_{T-1}. |
| Prior | X_{T-1} | This is the value | X_{T-1} = 0 |
| Biometric | | of the biometric | when the |
| Deviation | | deviation sensor | biometric monitor |
| Sensor | | value from a prior | 118 is first |
| Value | | iteration of the | initialized; |
| | | biometric | otherwise, |
| | | evaluation | X_{T-1} = X'_{T}. |
| | | module 216. | |
| Minimum | X'_{MIN} | This is a minimum | This value is a |
| Biometric | | threshold that the | constant and is |
| Deviation | | biometric deviation | initially determined |
| Threshold | | value should not go | based on the training |
| | | below. | by the training |
| | | | module 214. |
| Maximum | X'_{MAX} | This is a maximum | This value is a |
| Biometric | | threshold that the | constant and is |
| Deviation | | biometric deviation | initially determined |
| Threshold | | value. | based on the training |
| | | | by the training |
| | | | module 214. |
| Minimum | α_{MIN-COUNT} | This is a counter | 0 |
| Weighting | | that indicates the | |
| Factor | | number of times the | |
| Counter | | weighting factor | |
| | | has been less | |
| | | than or equal | |
| | | to the | |
| | | minimum | |
| | | weighting | |
| | | factor threshold. | |
| Maximum | α_{MAX-COUNT} | This is a counter that | 0 |
| Weighting | | indicates the number | |
| Factor | | of times the | |
| Counter | | weighting factor has | |
| | | been greater than or | |
| | | equal to the | |
| | | maximum | |
| | | weighting | |
| | | factor threshold. | |
| Minimum | α_{MIN-COUNT-T} | This is a minimum | This value is a |
| Weighting | | threshold that the | constant and is |
| Factor | | minimum weighting | initially determined |
| Counter | | factor counter | based on the training |
| Threshold | | threshold should | by the training |
| | | not go under. | module 214. |
| Maximum | α_{MAX-COUNT-T} | This is a maximum | This value is a |
| Weighting | | threshold that the | constant and is |
| Factor | | maximum weighting | initially determined |
| Counter | | factor counter | based on the training |
| Threshold | | threshold should | by the training |
| | | not exceed. | module 214. |
| Training | α_{TRAIN} | This is a Boolean | FALSE |
| Flag | | value that indicates | |
| | | whether the | |
| | | biometric monitor | |
| | | 118 should | |
| | | be trained. | |

As demonstrated by the foregoing table, one or more variables leveraged by the biometric monitor 118 are assigned an initialization value provided by the initialization biometric data 220. However, there may be instances where the value assigned to a given variable should be adjusted.

As discussed below with reference to FIGS. 4A-4E, one of the benefits provided by the disclosed biometric monitor 118 is that it can give advance warning to the user 120 if it determines that the biometric sensor data 224 indicates a likelihood that the user 120 is experiencing a health problem. However, to be more effective, the biometric monitor 118 can be trained and configured for the user's 120 unique physiology. Accordingly, in one embodiment, the biometric monitor 118 executes a biometric training module 214 that monitors for biometric sensor data 224 coming from the biometric sensor 116 during a predetermined time period (e.g., 30 minutes, an hour, one day, etc.) and stores various values as the trained biometric data 222.

During this predetermined time period, the biometric monitor 118 may obtain the trained biometric data 222 by executing the biometric training module 214 in conjunction with the biometric evaluation module 216, which, as discussed below, is configured to obtain the biometric deviation sensor value (e.g., X′_{T}). Furthermore, and during this predetermined time period, the biometric training module 214 may determine and/or record median values for the highs and lows of the biometric deviation sensor value, which are then stored as the maximum biometric deviation threshold and the minimum biometric deviation threshold, respectively. Similarly, the biometric training module 214 monitors the execution of the biometric evaluation module 216 and records median values corresponding to the minimum weighting factor counter threshold (e.g., α_{MIN-COUNT-T}) and the maximum weighting factor counter threshold (e.g., α_{MAX-COUNT-T}). The values acquired during this training period may then be stored as the trained biometric data 222. At the expiration of this predetermined time period, the biometric monitor 118 may communicate a message to the user 120, via the wearable computing device 104, that the biometric monitor 118 is configured to normally monitor the user 120.

In normal operation (e.g., outside of the training operations), the biometric monitor 118 executes a biometric evaluation module 216 configured to obtain the biometric deviation data 226, which includes one or more of the values described in Table 1 above. These values include, but are not limited to, the biometric deviation sensor value (e.g., X′_{T}), the weighting factor (e.g., α), the minimum weighting factor counter (e.g., α_{MIN-COUNT}), the maximum weighting factor counter (e.g., α_{MAC-COUNT}), and other such values or combination of values. In obtaining the biometric deviation data 226, the biometric evaluation module 216 executes the biometric evaluation logic 230, the operations of which are discussed with reference to FIGS. 3-4E below.

In one embodiment, the biometric evaluation module 216 determines the biometric deviation data 226 based on the biometric sensor data 224, which the biometric monitor 118 may be configured to continuously sample and/or receive while in operation. For example, the biometric monitor 118 may receive heart rate measurements from the biometric sensor 116 at a rate of one measurement every five seconds (e.g., 12 heart rate measurements per minute). Further still, this rate may be configurable, such that the biometric monitor 118 may be configured to receive the biometric sensor data 224 more or less frequently. In this manner, the biometric sensor data variable (e.g., X_{T}) may be assigned a new biometric sensor value, and a new biometric deviation sensor value (e.g., X′_{T}) may be determined, each time a biometric sensor measurement (e.g., a heart rate measurement) is received from the biometric sensor 116.

Should the biometric evaluation module 216 obtain biometric deviation data 226 that indicates the user 120 is experiencing a health problem, or a potential health problem, the biometric evaluation module 216 may execute a notification module 218 that provides one or more notifications accordingly. For example, and in one embodiment, the notification module 218 is configured with a first set of instructions corresponding to the condition where the biometric deviation sensor value is equal to or less than the minimum biometric deviation sensor value threshold and a second set of instructions corresponding to the condition where the biometric deviation sensor value is equal to or greater than the maximum biometric deviation sensor value threshold. The first and second set of instructions may be different, such as the messages or notifications that the biometric monitor 118 communicates to the wearable computing device 104. In this manner, the biometric monitor 118 can be configured to respond differently to different conditions being experienced by the user 120.

In addition, the biometric monitor 118 stores various values in Table 1 as the biometric re-training data 228 based on the execution of the biometric evaluation logic 230 by the biometric evaluation module 216. As the biometric monitor 118 may record one or more false positives and/or false negatives, these detected instances may be stored as the biometric re-training data 228. Examples of values that may be stored as the biometric re-training data 228 include, but are not limited to, the minimum weighting factor counter (e.g., α_{MIN-COUNT}), the maximum weighting factor counter (e.g., α_{MAX-COUNT}), and the training flag (e.g., α_{TRAIN}). The biometric monitor 118 may identify and/or record false positives and/or false negatives because one or more values have made the biometric monitor 118 too sensitive with respect to the changes in the biometric sensor data 224. Thus, when a given condition has been met that indicates that the biometric monitor 118 is too sensitive, the biometric evaluation module 216 may invoke the biometric training module 214 to re-train the biometric monitor 118 to better accommodate the user's 120 physiology.

FIG. 3 illustrates a method 302, according to an example embodiment, implemented by the biometric monitor 118 of FIG. 2 for notifying a user 120 of a potential health problem. The method 302 is implemented by one or more of the components of the biometric monitor 118 illustrated in FIG. 2 and is discussed by way of reference thereto.

As discussed above, the biometric monitor 118 initially executes the initialization module 212, which loads various values from the initialization biometric data 220 (Operation 304). The biometric monitor 118 may then enter a training phase, such as by executing the biometric training module 214, during which one or more variables are assigned values based on the training.

The biometric monitor 118 may then execute the biometric evaluation module 216 to begin receiving and evaluating the biometric sensor data 224 (Operation 306). During execution of the biometric evaluation module 216, the biometric evaluation module 216 may determine whether to re-train the biometric monitor 118 (Operation 308). In addition, the operations of the biometric monitor 118 include determining one or more of the biometric deviation data 226 (Operation 310). Finally, the operation of the biometric monitor 118 may include determining whether to notify the user 120 based on the determined biometric deviation data 226 (Operation 312). Operation of the biometric monitor 118 may then return to Operation 306, where the biometric monitor 118 may then receive new or additional biometric sensor data 224, which it then uses to re-determine the biometric deviation data 226.

FIGS. 4A-4E further illustrate a method 402, according to an example embodiment, implemented by the biometric monitor 118 of FIG. 2 for notifying a user 120 of a potential health problem. The method 402 is implemented by one or more components of the biometric monitor 118 and is discussed by way of reference there to.

Referring to FIG. 4A, the biometric monitor 118 initially receives an initialization instruction to initialize (Operation 404). This instruction may come in the form of electric power being applied to the biometric monitor 118, which causes a bootloader or other firmware-level code to initialize the biometric monitor 118.

In response to being initialized, the biometric monitor 118 then executes the initialization module 212, which causes one or more of the variables shown in Table 1 above to be assigned a preconfigured value stored in the initialization biometric data 220 (Operation 406). The initialization process may also include training the biometric monitor 118 with user-specific biometric sensor data 224 obtained from the biometric sensor 116. During the training of the biometric monitor 118, the biometric monitor 118 may forego one or more operations, such as the disclosed notification operations discussed further below.

When the biometric monitor 118 is placed into normal operation (e.g., non-training operation), the biometric monitor 118 then receives biometric sensor data 224 from the biometric sensor 116 (Operation 410). The biometric monitor 118 then determines whether the received biometric sensor data 224 is out of range with respect to the minimum biometric sensor data threshold or the maximum biometric sensor data threshold (e.g., whether X_{T}<X_{MIN} or X_{T}>X_{MAX}) (Operation 412). If this determination is made in the affirmative (e.g., the YES branch of Operation 412), then the method 402 proceeds to Operation 414. If this determination is made in the negative (e.g., the NO branch of Operation 412), then the method 402 proceeds to Operation 420 illustrated in FIG. 4B.

Referring to Operation 414, the biometric monitor 118 then determines whether the weighting factor (α) is less than the maximum weighting factor threshold (α_{MAX}) (Operation 414). If this determination is made in the affirmative (e.g., the YES branch of Operation 414), then the method 402 proceeds to Operation 416 of FIG. 4B. If this determination is made in the negative (e.g., the NO branch of Operation 414), then the method 402 also proceeds to Operation 420 of FIG. 4B.

Referring to FIG. 4B, where the method 402 is proceeding from the YES branch of Operation 414, the biometric monitor 118 increments the weighting factor (α) by the weighting factor increment (α_{INCREMENT}) (Operation 416). In this embodiment, the weighting factor is being incremented because it means that the current biometric sensor data (X_{T}) is out of range, and that the current biometric sensor data (X_{T}) should be given more emphasis than the prior biometric deviation sensor value (X_{T-1}). As more biometric sensor data values are out of range and the weighting factor (α) increases, there is more emphasis on the current biometric sensor data value (X_{T}).

In contrast, where the method 402 is proceeding from the NO branch of either Operation 414 or Operation 412, the biometric monitor 118 determines whether the weighting factor (α) is greater than the minimum weighting factor threshold (α_{MIN}) (Operation 420). If this determination is made in the affirmative (e.g., the YES branch of Operation 420), the biometric monitor 118 then decreases the weighting factor (α) by the weighting factor decrement (α_{DECREMENT}) (Operation 422). If this determination is made in the negative (e.g., the NO branch of Operation 420), then the method 402 proceeds to Operation 418.

From either Operation 416, Operation 420, or Operation 422, the biometric monitor 118 then determines whether the weighting factor (α) is less than or equal to the minimum weighting factor threshold (α_{MIN}) (Operation 418). If this determination is made in the affirmative (e.g., the YES branch of Operation 418), the biometric monitor 118 then increases the minimum weighting factor counter (α_{MIN-COUNT}). In one embodiment, the biometric monitor 118 increases the minimum weighting factor counter by one. The method 402 then proceeds to Operation 432 illustrated in FIG. 4C.

Alternatively, where the weighting factor (a) is not less than the minimum weighting factor threshold (α_{MIN}) (e.g., the NO branch of Operation 418), the biometric monitor 118 then determines whether the weighting factor (α) is greater than or equal to the maximum weighting factor threshold (α_{MAX}) (Operation 424). If this determination is made in the affirmative (e.g., the YES branch of Operation 424), then the method 402 proceeds to Operation 428 illustrated in FIG. 4C. If this determination is made in the negative (e.g., the NO branch of Operation 424), then the method 402 proceeds to Operation 442 illustrated in FIG. 4D. In effect, Operation 418 and Operation 424 determine whether the weighting factor (α) is out of range with its expected values.

Referring to FIG. 4C, at Operation 428, the biometric monitor 118 increases the maximum weighting factor counter (α_{MAX-COUNT}) (Operation 428). At Operations 430 and 432, the biometric monitor 118 determines whether the maximum weighting factor counter (α_{MAX-COUNT}) or the minimum weighting factor counter (α_{MIN-COUNT}) have exceeded their respective thresholds. These determinations (e.g., Operation 430 and Operation 432) are made because it indicates to the biometric monitor 118 whether the weighting factor is being adjusted (e.g., through increments or decrements) too frequently. If either of these determinations are made in the affirmative (e.g., the YES branch of Operation 430 or the YES branch of Operation 432), then the method 402 proceeds to Operation 434. Depending on whether the method 402 is proceeding from Operation 424 or Operation 426, if one of these determinations is made in the negative (e.g., the NO branch of Operation 430 or the NO branch of Operation 432), then the method 402 proceeds to FIG. 4D.

At Operation 434, the biometric monitor 118 determines whether the training flag (α_{TRAIN}) is TRUE or FALSE (Operation 434). The training flag is one mechanism that the biometric monitor 118 leverages to indicate whether further or additional training should be performed on the biometric monitor 118.

Where Operation 434 is determined in the affirmative (e.g., the YES branch of Operation 434), the biometric monitor 118 then executes the biometric training module 214 to conduct further training of the biometric monitor 118 and the various variables listed in Table 1 above. Where Operation 434 is determined in the negative (e.g., the NO branch of Operation 434), the method 402 proceeds to Operation 438.

As discussed above, the re-training of the biometric monitor 118 may include establishing new values for such variables as the minimum weighting factor counter threshold (α_{MIN-COUNT-T}), the maximum weighting factor counter threshold (α_{MAX-COUNT-T}), the minimum biometric deviation threshold (X′_{MIN}), the maximum biometric deviation threshold (X′_{MAX}), and other such values or combinations thereof. In one embodiment, biometric training module 214 performs the training of the biometric monitor 118 by executing the biometric evaluation 216, but omits one or more operations that would be performed in response to a determination that the user 120 is having a potential health problem (e.g., one or more of the notification operations). Furthermore, the training of the biometric monitor 118 may occur within a predetermined time period such that the biometric monitor 118 returns to “normal” operation after the expiration of such predetermined time period. The method 402 then proceeds to Operation 438.

At Operation 438, the biometric monitor 118 sets the value of the training flag (α_{TRAIN}) to TRUE. The biometric monitor 118 then logs the status of the training flag (α_{TRAIN}), which may also include logging the status of other variables of the biometric monitor 118, as well as the time and/or date when the training flag was set to TRUE.

Referring to FIG. 4D, the method 402 may proceed to FIG. 4D from the operations shown in FIG. 4C or through Operation 424 illustrated in FIG. 4B. With regard to Operation 424, the method 402 proceeds to FIG. 4D (and Operation 442) in response to a determination that the weighting factor (α) is not greater than or equal to the maximum weighting factor threshold (α_{MAX}) (e.g., α<α_{MAX}). At Operation 442, the biometric monitor 118 sets the value of the training flag (α_{TRAIN}) to FALSE (Operation 442). This operation is performed to account for the possibility that the training flag (α_{TRAIN}) may have been set to TRUE in a prior execution of the biometric evaluation module 216.

The biometric monitor 402 then sets the values of the minimum weighting factor counter (α_{MIN-COUNT}) (Operation 444) and the maximum weighting factor counter (α_{MAX-COUNT}) (Operation 446). In one embodiment, Operation 444 is implemented as: α_{MIN-COUNT}=max(0, α_{MIN-COUNT}−1). Similarly, in one embodiment, Operation 446 is implemented as: α_{MAX-COUNT}=max(0, α_{MAX-COUNT}−1). The biometric monitor 118 may perform these operations (e.g., Operations 442-446) to account for the instances where the biometric evaluation module 216 is performing as expected with few or no instances of the weighting factor (α) exceeding its minimum or maximum threshold.

At Operation 448, the biometric monitor 118 then determines the biometric deviation sensor value (X′_{T}) (Operation 448). In one embodiment, the biometric deviation sensor value (X′_{T}) is a summation of a weighted, current biometric sensor data (X_{T}) and a weighted, biometric deviation sensor value from a prior measurement (X_{T-1}). In particular, this determination may be presented by:

X′_{T}=(α×X_{T})+((1−α)×X_{T-1})

As shown above, the weighting factor determines whether more emphasis is placed on the current (or most recent) value of the biometric sensor data 224 or whether more emphasis is placed on the prior (or cumulative previous) biometric deviation sensor values. The weighting factor helps the biometric monitor 118 determine whether there is an ongoing change in the user's 120 biometric measurements and whether the user 120 should be notified of such changes.

The method 402 the proceeds to Operation 450, where the current biometric deviation sensor value (X′_{T}) is stored in a “running buffer” of one or more prior deviation sensor values (e.g., X_{T-1}, X_{T-2}, X_{T-3}, etc.). This running buffer is represented as the biometric deviation data 226 of FIG. 2. Furthermore, in alternative embodiments, the biometric monitor 118 may employ different weighting factors to account for different sets of timescales. In these alternative embodiments, a weighting factor corresponds to a particular timescale (e.g., a first weighting factor corresponds to a near-immediate timescale, a second weighting factor corresponds to a 4-measurement timescale, a third weighting factor corresponds to a 30-measurement timescale, and so forth). One of ordinary skill in the art will also appreciate that corresponding changes to the biometric evaluation logic 230 (e.g., as illustrated in FIGS. 4A-4E) would also be implemented. In this manner, the biometric monitor 118 can monitor the user's 120 biometric measurements over different time periods.

Referring to FIG. 4E, the biometric monitor 118 then performs various determinations as to whether the user 120 should be notified about changes in the biometric deviation sensor value. At Operation 452, the biometric monitor 118 determines whether biometric deviation sensor value (X′_{T}) is less than the minimum biometric deviation threshold (X′_{MIN}) (Operation 452). Where this determination is made in the affirmative (e.g., the YES branch of Operation 452), the biometric monitor 118 executes notification logic associated with the minimum biometric deviation threshold (Operation 454). For example, the biometric monitor 118 may execute the notification module 218, which notifies the user 120 accordingly. This notification may signal to the user 120 that his or her focal attention is low (e.g., where the biometric sensor data 224 is brain activity or eye focus) or it may communicate a message to an emergency provider that the user is experiencing a heart condition (e.g., where the biometric sensor data 224 is heart rate). The method 402 then proceeds to Operation 460.

Where Operation 452 is determined in the negative (e.g., the NO branch of Operation 452), the method 402 proceeds to Operation 456, where the biometric monitor 118 determines whether the biometric deviation sensor value (X′_{T}) is greater than the maximum biometric deviation threshold (X′_{MAX}) (Operation 456). Where this determination is made in the affirmative (e.g., the YES branch of Operation 456), the biometric monitor 118 executes notification logic associated with the maximum biometric deviation threshold (Operation 458). For example, the biometric monitor 118 may execute the notification module 218, which notifies the user 120 accordingly. This notification may signal to the user 120 that his or her heart rate is abnormally fast and/or it may communicate a message to an emergency provider that the user is experiencing a heart condition (e.g., where the biometric sensor data 224 is heart rate). The notification performed by the notification module 218 can be further customized and/or tailored to account for other physiological factors of the user 120, such as the user's 120 age, weight, gender, or other such factors and/or combination of factors.

Where Operation 456 is determined in the negative (e.g., the NO branch of Operation 456), the method proceeds to Operation 460, where the biometric monitor 118 communicates the determined biometric deviation sensor value (Operation 460). In one embodiment, the biometric monitor 118 communicates the biometric deviation sensor value to the wearable computing device 104 via the communication interface 204. Additionally or alternatively, the biometric monitor 118 may communicate the biometric deviation sensor value to other devices and/or systems, such as the server 112 illustrated in FIG. 1.

In this manner, the biometric monitor 118 is configured to predict whether a user 120 is experiencing a potential health problem based on changes to detected biometric measurement. Furthermore, the biometric 118 may be configured with instructions as to how the user 120 should be notified based on these detected changes. In addition, the biometric monitor 118 may be further trained to account for the user's 120 particular physiological so as to reduce the potential for false positives and/or false negatives. Furthermore, the operations performed by the biometric monitor 118 are fast and light-weight, which are well suited for mobile and embedded deployment. In particular, the biometric monitor 118 can be deployed with other CPU- and memory-intensive processes with less impact than alternative sensors with computations in the frequency domain or those that accumulate longer time series and store them in a memory buffer. This is technically beneficial because it means that the biometric monitor 118 can be used in a device, such as the wearable computing device 104, where computing resources (e.g., electric power, CPU cycles, machine-readable memory, etc.) are valued at a premium and are generally needed to perform more intensive computing operations. Furthermore, as the disclosed biometric monitor 118 has a small footprint, both physically and computationally, it can be embedded within the wearable computing device 104 without impacting physical comfort or computational abilities. Thus, the biometric sensor 118 has a number of technical benefits, both physically and computationally.

### Modules, Components, and Logic

Certain embodiments are described herein as including logic or a number of components, modules, or mechanisms. Modules may constitute either software modules (e.g., code embodied on a machine-readable medium) or hardware modules. A “hardware module” is a tangible unit capable of performing certain operations and may be configured or arranged in a certain physical manner. In various example embodiments, one or more computer systems (e.g., a standalone computer system, a client computer system, or a server computer system) or one or more hardware modules of a computer system (e.g., a processor or a group of processors) may be configured by software (e.g., an application or application portion) as a hardware module that operates to perform certain operations as described herein.

In some embodiments, a hardware module may be implemented mechanically, electronically, or any suitable combination thereof. For example, a hardware module may include dedicated circuitry or logic that is permanently configured to perform certain operations. For example, a hardware module may be a special-purpose processor, such as a Field-Programmable Gate Array (FPGA) or an Application Specific Integrated Circuit (ASIC). A hardware module may also include programmable logic or circuitry that is temporarily configured by software to perform certain operations. For example, a hardware module may include software executed by a general-purpose processor or other programmable processor. Once configured by such software, hardware modules become specific machines (or specific components of a machine) uniquely tailored to perform the configured functions and are no longer general-purpose processors. It will be appreciated that the decision to implement a hardware module mechanically, in dedicated and permanently configured circuitry, or in temporarily configured circuitry (e.g., configured by software) may be driven by cost and time considerations.

Accordingly, the phrase “hardware module” should be understood to encompass a tangible entity, be that an entity that is physically constructed, permanently configured (e.g., hardwired), or temporarily configured (e.g., programmed) to operate in a certain manner or to perform certain operations described herein. As used herein, “hardware-implemented module” refers to a hardware module. Considering embodiments in which hardware modules are temporarily configured (e.g., programmed), each of the hardware modules need not be configured or instantiated at any one instance in time. For example, where a hardware module comprises a general-purpose processor configured by software to become a special-purpose processor, the general-purpose processor may be configured as respectively different special-purpose processors (e.g., comprising different hardware modules) at different times. Software accordingly configures a particular processor or processors, for example, to constitute a particular hardware module at one instance of time and to constitute a different hardware module at a different instance of time.

Hardware modules can provide information to, and receive information from, other hardware modules. Accordingly, the described hardware modules may be regarded as being communicatively coupled. Where multiple hardware modules exist contemporaneously, communications may be achieved through signal transmission (e.g., over appropriate circuits and buses) between or among two or more of the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may then, at a later time, access the memory device to retrieve and process the stored output. Hardware modules may also initiate communications with input or output devices, and can operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions described herein. As used herein, “processor-implemented module” refers to a hardware module implemented using one or more processors.

Similarly, the methods described herein may be at least partially processor-implemented, with a particular processor or processors being an example of hardware. For example, at least some of the operations of a method may be performed by one or more processors or processor-implemented modules. Moreover, the one or more processors may also operate to support performance of the relevant operations in a “cloud computing” environment or as a “software as a service” (SaaS). For example, at least some of the operations may be performed by a group of computers (as examples of machines including processors), with these operations being accessible via a network (e.g., the Internet) and via one or more appropriate interfaces (e.g., an Application Program Interface (API)).

The performance of certain of the operations may be distributed among the processors, not only residing within a single machine, but deployed across a number of machines. In some example embodiments, the processors or processor-implemented modules may be located in a single geographic location (e.g., within a home environment, an office environment, or a server farm). In other example embodiments, the processors or processor-implemented modules may be distributed across a number of geographic locations.

### Example Machine Architecture and Machine-Readable Medium

FIG. 5 is a block diagram illustrating components of a machine 500, according to some example embodiments, able to read instructions from a machine-readable medium (e.g., a machine-readable storage medium) and perform any one or more of the methodologies discussed herein. Specifically, FIG. 5 shows a diagrammatic representation of the machine 500 in the example form of a computer system, within which instructions 516 (e.g., software, a program, an application, an applet, an app, or other executable code) for causing the machine 500 to perform any one or more of the methodologies discussed herein may be executed. For example, the instructions may cause the machine to execute the method illustrated in FIG. 3 and FIGS. 4A-4E. Additionally, or alternatively, the instructions may implement one or more of the modules 208 illustrated in FIG. 2 and so forth. The instructions transform the general, non-programmed machine into a particular machine programmed to carry out the described and illustrated functions in the manner described. In alternative embodiments, the machine 500 operates as a standalone device or may be coupled (e.g., networked) to other machines. In a networked deployment, the machine 500 may operate in the capacity of a server machine or a client machine in a server-client network environment, or as a peer machine in a peer-to-peer (or distributed) network environment. The machine 500 may comprise, but not be limited to, a server computer, a client computer, a personal computer (PC), a tablet computer, a laptop computer, a netbook, a set-top box (STB), a personal digital assistant (PDA), an entertainment media system, a cellular telephone, a smart phone, a mobile device, a wearable device (e.g., a smart watch), a smart home device (e.g., a smart appliance), other smart devices, a web appliance, a network router, a network switch, a network bridge, or any machine capable of executing the instructions 516, sequentially or otherwise, that specify actions to be taken by machine 500. Further, while only a single machine 500 is illustrated, the term “machine” shall also be taken to include a collection of machines 500 that individually or jointly execute the instructions 516 to perform any one or more of the methodologies discussed herein.

The machine 500 may include processors 510, memory 530, and I/O components 550, which may be configured to communicate with each other such as via a bus 502. In an example embodiment, the processors 510 (e.g., a Central Processing Unit (CPU), a Reduced Instruction Set Computing (RISC) processor, a Complex Instruction Set Computing (CISC) processor, a Graphics Processing Unit (GPU), a Digital Signal Processor (DSP), an Application Specific Integrated Circuit (ASIC), a Radio-Frequency Integrated Circuit (RFIC), another processor, or any suitable combination thereof) may include, for example, processor 512 and processor 514 that may execute instructions 516. The term “processor” is intended to include multi-core processor that may comprise two or more independent processors (sometimes referred to as “cores”) that may execute instructions contemporaneously. Although FIG. 5 shows multiple processors, the machine 500 may include a single processor with a single core, a single processor with multiple cores (e.g., a multi-core process), multiple processors with a single core, multiple processors with multiples cores, or any combination thereof.

The memory/storage 530 may include a memory 532, such as a main memory, or other memory storage, and a storage unit 536, both accessible to the processors 510 such as via the bus 502. The storage unit 536 and memory 532 store the instructions 516 embodying any one or more of the methodologies or functions described herein. The instructions 516 may also reside, completely or partially, within the memory 532, within the storage unit 536, within at least one of the processors 510 (e.g., within the processor's cache memory), or any suitable combination thereof, during execution thereof by the machine 500. Accordingly, the memory 532, the storage unit 536, and the memory of processors 510 are examples of machine-readable media.

As used herein, “machine-readable medium” means a device able to store instructions and data temporarily or permanently and may include, but is not be limited to, random-access memory (RAM), read-only memory (ROM), buffer memory, flash memory, optical media, magnetic media, cache memory, other types of storage (e.g., Erasable Programmable Read-Only Memory (EEPROM)) and/or any suitable combination thereof. The term “machine-readable medium” should be taken to include a single medium or multiple media (e.g., a centralized or distributed database, or associated caches and servers) able to store instructions 516. The term “machine-readable medium” shall also be taken to include any medium, or combination of multiple media, that is capable of storing instructions (e.g., instructions 516) for execution by a machine (e.g., machine 500), such that the instructions, when executed by one or more processors of the machine 500 (e.g., processors 510), cause the machine 500 to perform any one or more of the methodologies described herein. Accordingly, a “machine-readable medium” refers to a single storage apparatus or device, as well as “cloud-based” storage systems or storage networks that include multiple storage apparatus or devices. The term “machine-readable medium” excludes signals per se.

The I/O components 550 may include a wide variety of components to receive input, provide output, produce output, transmit information, exchange information, capture measurements, and so on. The specific I/O components 550 that are included in a particular machine will depend on the type of machine. For example, portable machines such as mobile phones will likely include a touch input device or other such input mechanisms, while a headless server machine will likely not include such a touch input device. It will be appreciated that the I/O components 550 may include many other components that are not shown in FIG. 5. The I/O components 550 are grouped according to functionality merely for simplifying the following discussion and the grouping is in no way limiting. In various example embodiments, the I/O components 550 may include output components 552 and input components 554. The output components 552 may include visual components (e.g., a display such as a plasma display panel (PDP), a light emitting diode (LED) display, a liquid crystal display (LCD), a projector, or a cathode ray tube (CRT)), acoustic components (e.g., speakers), haptic components (e.g., a vibratory motor, resistance mechanisms), other signal generators, and so forth. The input components 554 may include alphanumeric input components (e.g., a keyboard, a touch screen configured to receive alphanumeric input, a photo-optical keyboard, or other alphanumeric input components), point based input components (e.g., a mouse, a touchpad, a trackball, a joystick, a motion sensor, or other pointing instrument), tactile input components (e.g., a physical button, a touch screen that provides location and/or force of touches or touch gestures, or other tactile input components), audio input components (e.g., a microphone), and the like.

In further example embodiments, the I/O components 550 may include biometric components 556, motion components 558, environmental components 560, or position components 562 among a wide array of other components. For example, the biometric components 556 may include components to detect expressions (e.g., hand expressions, facial expressions, vocal expressions, body gestures, or eye tracking), measure biosignals (e.g., blood pressure, heart rate, body temperature, perspiration, or brain waves), identify a person (e.g., voice identification, retinal identification, facial identification, fingerprint identification, or electroencephalogram based identification), and the like. The motion components 558 may include acceleration sensor components (e.g., accelerometer), gravitation sensor components, rotation sensor components (e.g., gyroscope), and so forth. The environmental components 560 may include, for example, illumination sensor components (e.g., photometer), temperature sensor components (e.g., one or more thermometer that detect ambient temperature), humidity sensor components, pressure sensor components (e.g., barometer), acoustic sensor components (e.g., one or more microphones that detect background noise), proximity sensor components (e.g., infrared sensors that detect nearby objects), gas sensors (e.g., gas detection sensors to detection concentrations of hazardous gases for safety or to measure pollutants in the atmosphere), or other components that may provide indications, measurements, or signals corresponding to a surrounding physical environment. The position components 562 may include location sensor components (e.g., a Global Position System (GPS) receiver component), altitude sensor components (e.g., altimeters or barometers that detect air pressure from which altitude may be derived), orientation sensor components (e.g., magnetometers), and the like.

Communication may be implemented using a wide variety of technologies. The I/O components 550 may include communication components 564 operable to couple the machine 500 to a network 580 or devices 570 via coupling 582 and coupling 572 respectively. For example, the communication components 564 may include a network interface component or other suitable device to interface with the network 580. In further examples, communication components 564 may include wired communication components, wireless communication components, cellular communication components, Near Field Communication (NFC) components, Bluetooth® components (e.g., Bluetooth® Low Energy), Wi-Fi® components, and other communication components to provide communication via other modalities. The devices 570 may be another machine or any of a wide variety of peripheral devices (e.g., a peripheral device coupled via a Universal Serial Bus (USB)).

Moreover, the communication components 564 may detect identifiers or include components operable to detect identifiers. For example, the communication components 564 may include Radio Frequency Identification (RFID) tag reader components, NFC smart tag detection components, optical reader components (e.g., an optical sensor to detect one-dimensional bar codes such as Universal Product Code (UPC) bar code, multi-dimensional bar codes such as Quick Response (QR) code, Aztec code, Data Matrix, Dataglyph, MaxiCode, PDF417, Ultra Code, UCC RSS-2D bar code, and other optical codes), or acoustic detection components (e.g., microphones to identify tagged audio signals). In addition, a variety of information may be derived via the communication components 564, such as, location via Internet Protocol (IP) geo-location, location via Wi-Fi® signal triangulation, location via detecting a NFC beacon signal that may indicate a particular location, and so forth.

### Transmission Medium

In various example embodiments, one or more portions of the network 580 may be an ad hoc network, an intranet, an extranet, a virtual private network (VPN), a local area network (LAN), a wireless LAN (WLAN), a wide area network (WAN), a wireless WAN (WWAN), a metropolitan area network (MAN), the Internet, a portion of the Internet, a portion of the Public Switched Telephone Network (PSTN), a plain old telephone service (POTS) network, a cellular telephone network, a wireless network, a Wi-Fi® network, another type of network, or a combination of two or more such networks. For example, the network 580 or a portion of the network 580 may include a wireless or cellular network and the coupling 582 may be a Code Division Multiple Access (CDMA) connection, a Global System for Mobile communications (GSM) connection, or other type of cellular or wireless coupling. In this example, the coupling 582 may implement any of a variety of types of data transfer technology, such as Single Carrier Radio Transmission Technology (1xRTT), Evolution-Data Optimized (EVDO) technology, General Packet Radio Service (GPRS) technology, Enhanced Data rates for GSM Evolution (EDGE) technology, third Generation Partnership Project (3GPP) including 3G, fourth generation wireless (4G) networks, Universal Mobile Telecommunications System (UMTS), High Speed Packet Access (HSPA), Worldwide Interoperability for Microwave Access (WiMAX), Long Term Evolution (LTE) standard, others defined by various standard setting organizations, other long range protocols, or other data transfer technology.

The instructions 516 may be transmitted or received over the network 580 using a transmission medium via a network interface device (e.g., a network interface component included in the communication components 564) and utilizing any one of a number of well-known transfer protocols (e.g., hypertext transfer protocol (HTTP)). Similarly, the instructions 516 may be transmitted or received using a transmission medium via the coupling 572 (e.g., a peer-to-peer coupling) to devices 570. The term “transmission medium” shall be taken to include any intangible medium that is capable of storing, encoding, or carrying instructions 516 for execution by the machine 500, and includes digital or analog communications signals or other intangible medium to facilitate communication of such software.

### Language

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Although an overview of the inventive subject matter has been described with reference to specific example embodiments, various modifications and changes may be made to these embodiments without departing from the broader scope of embodiments of the present disclosure. Such embodiments of the inventive subject matter may be referred to herein, individually or collectively, by the term “invention” merely for convenience and without intending to voluntarily limit the scope of this application to any single disclosure or inventive concept if more than one is, in fact, disclosed.

The embodiments illustrated herein are described in sufficient detail to enable those skilled in the art to practice the teachings disclosed. Other embodiments may be used and derived therefrom, such that structural and logical substitutions and changes may be made without departing from the scope of this disclosure. The Detailed Description, therefore, is not to be taken in a limiting sense, and the scope of various embodiments is defined only by the appended claims, along with the full range of equivalents to which such claims are entitled.

As used herein, the term “or” may be construed in either an inclusive or exclusive sense. Moreover, plural instances may be provided for resources, operations, or structures described herein as a single instance. Additionally, boundaries between various resources, operations, modules, engines, and data stores are somewhat arbitrary, and particular operations are illustrated in a context of specific illustrative configurations. Other allocations of functionality are envisioned and may fall within a scope of various embodiments of the present disclosure. In general, structures and functionality presented as separate resources in the example configurations may be implemented as a combined structure or resource. Similarly, structures and functionality presented as a single resource may be implemented as separate resources. These and other variations, modifications, additions, and improvements fall within a scope of embodiments of the present disclosure as represented by the appended claims. The specification and drawings are, accordingly, to be regarded in an illustrative rather than a restrictive sense.

We claim:

1. A biometric monitor for monitoring provided biometric sensor data, the biometric monitor comprising:
a machine-readable memory storing computer-executable instructions; and
at least one hardware processor in communication with the machine-readable memory that, when the computer-executable instructions are executed, configures the biometric monitor to:
receive biometric sensor data;
determine whether the biometric sensor data is out of an expected range;
in response to the determination that the biometric sensor data is out of an expected range, adjust a first weighting factor by a predetermined amount;
determine whether the first weighting factor is out of an expected range;
in response to the determination that the first weighting factor is out of the expected range, increment a counter associated with the first weighting factor;
compute a biometric deviation sensor value based on the first weighting factor, the received biometric sensor data, and a previously computed biometric deviation sensor value; and
communicate the computed biometric deviation sensor value via a communication interface communicatively coupled to the at least one hardware processor.

2. The biometric monitor of claim 1, wherein the first weighting factor is adjusted by incrementing the first weighting factor by the predetermined amount.

3. The biometric monitor of claim 1, wherein the first weighting factor is adjusted by decrementing the first weighting factor by the predetermined amount.

4. The biometric monitor of claim 1, wherein the determination that the first weighting factor is out of the expected range comprises comparing the first weighting factor with a minimum weighting factor threshold, and the counter is associated with the minimum weighting factor threshold.

5. The biometric monitor of claim 4, wherein the biometric monitor is further configured to:
compare the counter with a minimum counter threshold; and
based on the comparison of the counter with the minimum counter threshold, execute one or more training operations to train the biometric monitor based on further received biometric sensor data.

6. The biometric monitor of claim 1, wherein the determination that the first weighting factor is out of the expected range comprises comparing the first weighting factor with a maximum weighting factor threshold, and the counter is associated with the maximum weighting factor threshold.

7. The biometric monitor of claim 1, wherein the biometric monitor is further configured to:
determine whether the computed biometric deviation sensor value exceeds a maximum biometric deviation sensor value threshold;
determine whether the computer biometric deviation sensor value is less than a minimum biometric deviation sensor value threshold;
in response to the determination that the computed biometric deviation sensor value exceeds the maximum biometric deviation sensor value threshold, execute at least one notification operation associated with the maximum biometric deviation sensor value threshold; and
in response to the determination that the computed biometric deviation sensor value is less than the minimum biometric deviation sensor value threshold, execute at least one notification operation associated with the minimum biometric deviation sensor value threshold, the at least one notification operation associated with the minimum biometric deviation sensor value threshold being different than the at least one notification operation associated with the maximum biometric deviation sensor value threshold.

8. A method for monitoring provided biometric sensor data, the method comprising:
receiving, by at least one hardware processor, biometric sensor data;
determining, by at least one hardware processor, whether the biometric sensor data is out of an expected range;
in response to the determination that the biometric sensor data is out of an expected range, adjusting, by at least one hardware processor, a first weighting factor by a predetermined amount;
determining, by at least one hardware processor, whether the first weighting factor is out of an expected range;
in response to the determination that the first weighting factor is out of the expected range, incrementing, by at least one hardware processor, a counter associated with the first weighting factor;
computing, by at least one hardware processor, a biometric deviation sensor value based on the first weighting factor, the received biometric sensor data, and a previously computed biometric deviation sensor value; and
communicating, by at least one hardware processor, the computed biometric deviation sensor value via a communication interface communicatively coupled to the at least one hardware processor.

9. The method of claim 8, wherein the first weighting factor is adjusted by incrementing the first weighting factor by the predetermined amount.

10. The method of claim 8, wherein the first weighting factor is adjusted by decrementing the first weighting factor by the predetermined amount.

11. The method of claim 8, wherein the determination that the first weighting factor is out of the expected range comprises comparing the first weighting factor with a minimum weighting factor threshold, and the counter is associated with the minimum weighting factor threshold.

12. The method of claim 11, wherein the method further comprises:
comparing the counter with a minimum counter threshold; and
based on the comparison of the counter with the minimum counter threshold, executing one or more training operations to train a biometric monitor based on further received biometric sensor data.

13. The method of claim 8, wherein the determination that the first weighting factor is out of the expected range comprises comparing the first weighting factor with a maximum weighting factor threshold, and the counter is associated with the maximum weighting factor threshold.

14. The method of claim 8, further comprising:
determining whether the computed biometric deviation sensor value exceeds a maximum biometric deviation sensor value threshold;
determining whether the computer biometric deviation sensor value is less than a minimum biometric deviation sensor value threshold;
in response to the determination that the computed biometric deviation sensor value exceeds the maximum biometric deviation sensor value threshold, executing at least one notification operation associated with the maximum biometric deviation sensor value threshold; and
in response to the determination that the computed biometric deviation sensor value is less than the minimum biometric deviation sensor value threshold, executing at least one notification operation associated with the minimum biometric deviation sensor value threshold, the at least one notification operation associated with the minimum biometric deviation sensor value threshold being different than the at least one notification operation associated with the maximum biometric deviation sensor value threshold.

15. A machine-readable medium storing computer-executable instructions that, when executed by at least one hardware processor, causes a biometric monitor to perform a plurality of operations, the operations comprising:
receiving biometric sensor data;
determining whether the biometric sensor data is out of an expected range;
in response to the determination that the biometric sensor data is out of an expected range, adjusting a first weighting factor by a predetermined amount;
determining whether the first weighting factor is out of an expected range;
in response to the determination that the first weighting factor is out of the expected range, incrementing a counter associated with the first weighting factor;
computing a biometric deviation sensor value based on the first weighting factor, the received biometric sensor data, and a previously computed biometric deviation sensor value; and
communicating the computed biometric deviation sensor value via a communication interface communicatively coupled to at least one hardware processor.

16. The machine-readable medium of claim 15, wherein the first weighting factor is adjusted by incrementing the first weighting factor by the predetermined amount.

17. The machine-readable medium of claim 15, wherein the first weighting factor is adjusted by decrementing the first weighting factor by the predetermined amount.

18. The machine-readable medium of claim 15, wherein the determination that the first weighting factor is out of the expected range comprises comparing the first weighting factor with a minimum weighting factor threshold, and the counter is associated with the minimum weighting factor threshold.

19. The machine-readable medium of claim 15, wherein the plurality of operations further comprise:
comparing the counter with a minimum counter threshold; and
based on the comparison of the counter with the minimum counter threshold, executing one or more training operations to train a biometric monitor based on further received biometric sensor data.

20. The machine-readable medium of claim 15, wherein the plurality of operations further comprise:
determining whether the computed biometric deviation sensor value exceeds a maximum biometric deviation sensor value threshold;
determining whether the computer biometric deviation sensor value is less than a minimum biometric deviation sensor value threshold;
in response to the determination that the computed biometric deviation sensor value exceeds the maximum biometric deviation sensor value threshold, executing at least one notification operation associated with the maximum biometric deviation sensor value threshold; and
in response to the determination that the computed biometric deviation sensor value is less than the minimum biometric deviation sensor value threshold, executing at least one notification operation associated with the minimum biometric deviation sensor value threshold, the at least one notification operation associated with the minimum biometric deviation sensor value threshold being different than the at least one notification operation associated with the maximum biometric deviation sensor value threshold.
